(19) United States
(12) Patent Application Publication
(10) Pub. No.: US 2014/0257161 A1
(43) Pub. Date: Sep. 11, 2014

(54) Ankle Splint

(71) Applicant: William Russel Compton, Louisville, KY (US)

(72) Inventor: William Russel Compton, Louisville, KY (US)

(21) Appl. No.: 13/786,777

(22) Filed: Mar. 6, 2013

(51) Int. Cl. A61F 5/058

(52) U.S. Cl. CPC A61F 5/0585
USPC 602/27

(57) ABSTRACT

The present disclosure provides for a splint that is placed onto the lower extremity of the human body in the event of a bone fracture. The present disclosure partially encloses the lower extremity that is injured due to the bone fracture, as a means of providing the injured area ample room to swell after injury without affecting the structure and fit of the splint. Furthermore, the disclosure is comprised allows for a polyurethane foam to fill a reservoir via ports found along the side of the disclosure, which then acts as a support and immobilization means to the affected area. Additionally, the splint design provides for a removable liner that allows for a lightweight support structure to be used post operation.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### FIELD OF THE INVENTION

The present invention relates generally to a splint that is placed onto and partially encloses a lower extremity of a human body in the event of a bone fracture and is intended to immobilize the joint in a reduced state until such a time that the fracture can be surgically corrected.

### BACKGROUND OF THE INVENTION

A splint is generally understood to be a support that holds and protects a body part after an injury. The present invention relates to a splint that is placed onto and partially encloses a lower extremity of the human body in the event of a fracture. The splint is intended to immobilize the injured joint in a reduced state until such time that the fracture can be healed and/or surgically corrected.

Currently, when a patient sustains an ankle injury or fracture, the ankle is placed in a temporary rigid and fully enclosed splint composed of multiple layers of cotton padding, rigid fiberglass and elastic bandages. Initially, the fracture is aligned (or reduced), which is then followed by the placement of said temporary rigid splint. The temporary rigid splint application requires one person to hold the limb in alignment while a second person applies a protective sock-like liner, followed by several layers of cotton padding, followed by a longitudinal section of rolled fiberglass, then a second section of fiberglass may be added as a stirrup to hold the fracture in place. This is followed by the application of elastic bandages which keep the construct in place while the rolled fiberglass dries. Furthermore, the physician must hold the ankle in place while the fiberglass dries to ensure that reduction of the fracture is maintained. This current practice is time-consuming, requires two people to accomplish, and is heavy and uncomfortable for the patient.

Furthermore, the initial rigid splint is temporary and it will be discarded after definitive surgical correction takes place, usually within a week, only to have a solid ‘traditional’ fiberglass cast applied. This fiberglass cast, too, is temporary and will be removed after the fracture is healed. Only then will the patent be placed in a semi-rigid ‘walking cast’ for rehabilitation of the fracture. Thus, the current practice utilizes three different apparatus for one injury, which adds additional cost to treatment of these types of injuries.

The present invention improves on the current state of the art in the following ways: (1) It is lighter and more comfortable for the patient; (2) It can be used from the time of initial injury, after surgical correction, and through the rehabilitation period, thus requiring only one apparatus for treatment; (3) It is it extremely cost efficient; (4) It is semi-enclosed and only covers the posterior aspect of the lower extremity, providing room for the injured area to swell after injury; (5) It only requires one person for application; and (6) It is faster and easier to apply when compared to what is generally used currently.

The splint described in the present invention is designed such that it can be used on the injured patient even during the post-operative period until the fracture is deemed healed, and can be further used in the rehabilitative period as a semi-rigid support.

### BRIEF SUMMARY OF THE INVENTION

It is an object of the present invention to overcome the problems, obstacles and deficiencies of the prior art.

It is also an object of the present invention to create a splint that is placed onto a lower extremity of a human body in the event of a bone fracture.

It is an object of the present invention to create a splint that only partially encloses a lower extremity of a human body in the event of a bone fracture.

It is also an object of the invention to immobilize the injured lower extremity by placing it first in a soft inner expandable boot containing ports along its perimeter for introduction of a polyurethane foam that expands around the injured site and holds the injured site firmly in place.

It is an object of the invention to place the soft inner boot into an outer rigid boot which provides additional support for the injured lower extremity.

Many other embodiments will be readily apparent to those of ordinary skill in the art upon viewing the drawings and reading the detailed description hereafter.

### BRIEF DESCRIPTION OF THE DRAWINGS

The subject matter, which is regarded as the invention, is particularly pointed out and distinctly claimed in the claims at the conclusion of the specification. The foregoing and other features and advantages of the invention will be apparent from the following detailed description taken in conjunction with the accompanying drawings in which:

FIG. 1 illustrates a schematic representation of one embodiment of the present invention.

FIG. 2 illustrates a schematic representation of another embodiment of the present invention.

FIG. 3 illustrates a schematic representation of yet another embodiment of the present invention.

### DETAILED DESCRIPTION OF THE INVENTION

It should be understood that the embodiments detailed above are only some examples of the many possible forms that the invention can use. In general, statements made in the specification of the present application do not necessarily limit any of the claims of the invention. Moreover, some statements may apply to some inventive features but not to others. In general, unless otherwise indicated, singular statement may be in the plural and vice-versa with no loss of generality.

The present invention relates generally to a splint that is placed onto a lower extremity of a human body in the event of a bone fracture. The splint only partially encloses the lower extremity that is injured due to a bone fracture.

The splint is intended to immobilize the injured joint in a reduced state until such time that the fracture can be healed and/or surgically corrected.

The splint described in the present invention is designed such that it can be used on the injured patient even during the post-operative period until the fracture is deemed healed, and can be further used in the rehabilitative period as a semi-rigid support.

The present invention improves on the prior art in that it is semi-enclosed and only covers the posterior part of the injured extremity, thus providing room for the injured area to swell after injury without affecting the structure and fit of the splint. In general, a fracture site experiences maximum swelling within 24-48 hours of the injury. Due to the semi-enclosed design of the present invention, any swelling to the fracture site moves to the open or unenclosed area of the lower extremity. The actual fracture site is immobilized by the splint and is not impacted by the swelling. Similarly, any reduction in swelling also fails to impact the fit and comfort of the splint.

The splint described in the present invention consists an external rigid boot and an internal soft boot, which is also sometimes called the internal liner. This internal boot is consists of 2 layers seamed together at the edges to form an expandable compartment. The internal boot may be composed of 2 layers of neoprene or silicone rubber or similar soft material for protection of the patient's skin. In the present invention, the seamed inner soft boot is pre-formed to approximate the size and shape of the injured lowered extremity and only covers the posterior aspect of the injured extremity. The inner boot is also equipped with ports along its perimeter for injection of a polyurethane foam, which flows into the inner boot and solidifies around the injured extremity, thereby immobilizing the injured area. The polyurethane foam is supplied with the splint in a separate pressurized container.

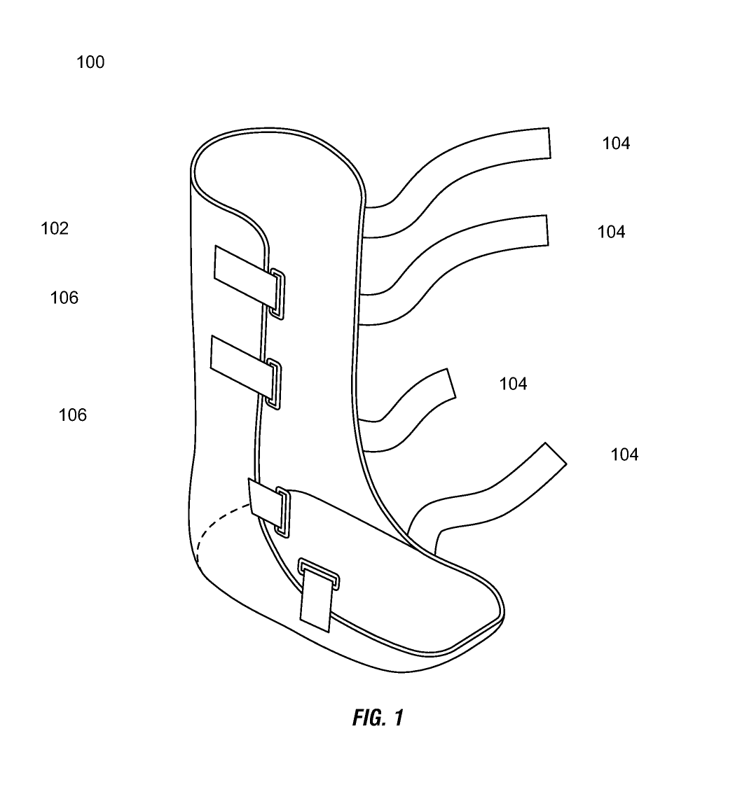

Referring now to FIG. 1, the schematic view 100 of an exemplary embodiment incorporating the design of the rigid external boot of the splint is depicted. At 102, the splint's rigid exterior structure is shown. The rigid external boot 102 will be held in place via a plurality of Velcro © straps 104 transversely along external boot 102 anterior aspect. In an alternative embodiment of the present disclosure, rigid external boot 102 will be composed of injected molded high density polyethylene (HDPE) such as Dow Chemical HDPE35060E or similar. Furthermore, rigid external boot 102 will have Velcro © “double faced looped” straps transversely across its anterior surface. At 106, the fill ports for injecting a polyurethane foam is depicted. The polyurethane foam is injected into the silicone bladder of the inner layer via ports 106 as a means for stabilizing and conforming to the injured foot. In one embodiment of the present invention, the polyurethane foam is initially in liquid form allowing it to flow around the fracture site and fill the reservoir created in the silicone bladder. Once cured, the polyurethane foam will solidify and will serve to immobilize the injury. The solidification and curing of the polyurethane foam takes approximately five minutes.

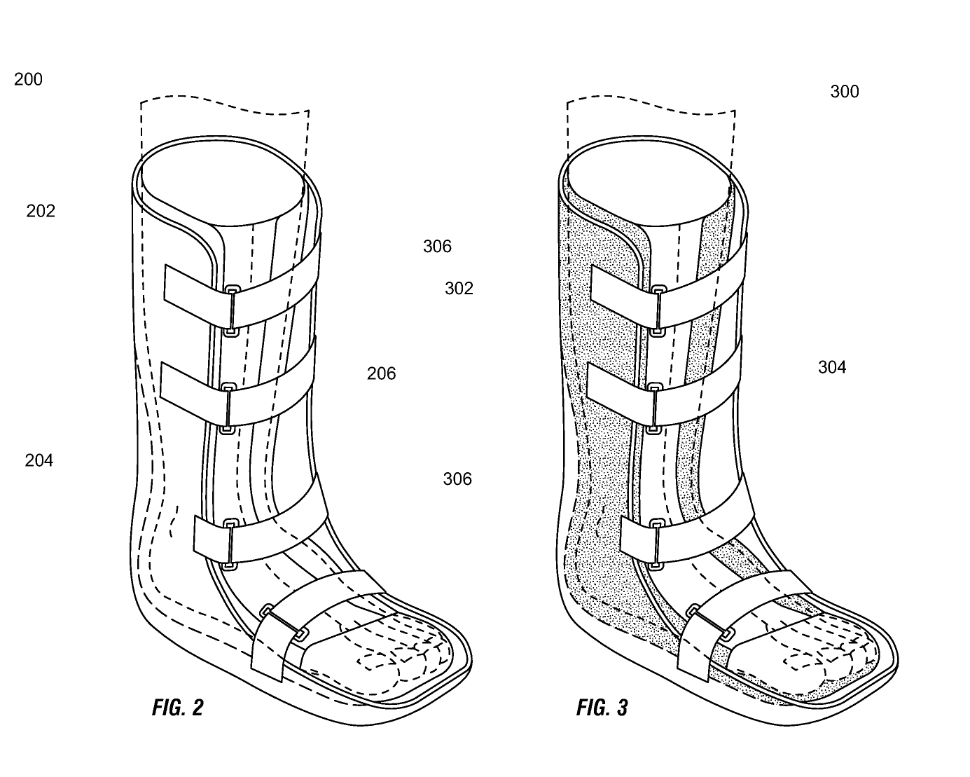

Referring now to FIG. 2, the schematic view 200 of yet another exemplary embodiment incorporating various splint design components of the present disclosure is depicted. At 202, the rigid external boot is depicted. In the particular embodiment, inner liner of the present disclosure is shown. The inner liner of the present disclosure is an expandable bladder with two layers of silicone bonded at the seam with inert silicone cement. In one embodiment of the present disclosure the inner liner is composed of FDA compliant silicone rubber. At 204, the external layer of the inner liner is shown. External Layer 204 of the inner liner has a plurality of ports along the lateral side. Said ports found along the lateral side are designated as the area in which the bladder of the inner layer can be filled with polyurethane foam. The polyurethane foam is initially in liquid form allowing it to flow around the fracture site and fill the reservoir created in the silicone bladder of the inner liner. Once the polyurethane foam is allowed to flow around the injury, the inner liner or boot is secured around the lower extremity using a Velcro 0 closure 206 found longitudinally along the anterior aspect.

Referring now to FIG. 3, a schematic view 300 of another exemplary embodiment incorporating various splint design components of the present disclosure is depicted. At 302, the inner liner of the disclosure that contacts the skin is shown. Inner liner 302, has a smooth, brushed surface to minimize friction and enhance comfort to the individual. Inner liner 304 is a light weight, flexible and durable material that can be placed into and removed easily from the external boot. At 306, fill ports used for injection polyurethane foam is depicted. Fill ports 306 serve the function as described above in FIG. 1.

Post-operatively, the inner liner can be reapplied, or an additional inner liner can be created if needed. The splint system can continue to be utilized in the previously described manner. After the operatively fixed fracture has healed, the inner liner can be removed, and the external boot can be fitted with the standard air pillows and cushioned liner to allow for support during rehabilitation.

It should be emphasized that the above-described embodiment of the invention is merely a possible example of implementations set forth for a clear understanding of the principles of the invention. Variations and modifications may be made to the above-described embodiment of the invention without departing from the spirit and principles of the invention. All such modifications and variations are intended to be included herein within the scope of the invention and protected by the following claims.

What is claimed is:

1. A semi-enclosed orthopedic splint system composed of two parts: a rigid outer shell/boot, and a semi-enclosed inner expandable liner or boot consisting of two layers of neoprene (or similar material), wherein the inner liner has ports along a perimeter for the injection of a polyurethane foam, and wherein, the splint, once formed, will accomplish adequate immobilization to maintain reduction of a fracture throughout an injury period.

2. The Orthopedic splint of claim 1, wherein the splint can be removed and re-applied post-operatively, and can continue to be used in a rehabilitation period by simply removing the inner liner and replacing it with a readily available soft, padded liner. The splint will be faster, easier, lighter weight and only require one practitioner to apply.
